(19) United States
(12) Patent Application Publication
(10) Pub. No.: US 2024/0325359 A1
(43) Pub. Date: Oct. 3, 2024

(54) ACAT1 INHIBITOR AND METHOD OF USE IN THE TREATMENT OF DISEASE

(71) Applicant: Trustees of Dartmouth College, Hanover, NH (US)

(72) Inventors: Ta Yuan CHANG, Etna, NH (US); Catherine C.Y. CHANG, Etna, NH (US); Adrianna DE LA TORRE, Lyme, NH (US)

(21) Appl. No.: 18/599,793

(22) Filed: Mar. 8, 2024

Related U.S. Application Data
(60) Provisional application No. 63/454,767, filed on Mar. 27, 2023.

(51) Int. Cl. A61K 31/44; A61K 9/51; C07D 213/75

(52) U.S. Cl. CPC A61K 31/44; A61K 9/5123; C07D 213/75

(57) ABSTRACT

A high potency, brain-permeable compound that inhibits Acyl-CoA:Cholesterol Acyltransferase 1 (ACAT1) activity is provided as are pharmaceutical compositions and methods for treating a disease, disorder, or condition mediated by ACAT1.

This application claims the benefit of priority from U.S. Provisional Application Ser. No. 63/454,767 filed Mar. 27, 2023, the teachings of which are herein incorporated by reference in their entirety.

### INTRODUCTION

This invention was made with government support under Grant Number AG063544 awarded by the National Institutes of Health. The government has certain rights in the invention.

### BACKGROUND

Chronic neuroinflammation is a hallmark of late onset Alzheimer's disease (LOAD) and in many other neurodegenerative diseases, as well as in aging. In the central nervous system (CNS), the Toll-Like Receptor 4 (TLR4) is expressed in microglia, astrocytes, oligodendrocytes, and neurons. It is a transmembrane receptor protein that recognizes diverse pathogen-derived ligands including lipopolysaccharides (LPS), and various tissue damage-related ligands including oligomeric amyloid peptide fragment Aβ₁₋₄₂, heat-shock proteins (especially HSP60 and HSP70), high mobility group box 1 (HMGB1), hyaluronic acid, fibronectin, galectin-3, and the like. TLR4 is a LOAD susceptibility gene. In LOAD, aging, and several neurodegenerative diseases, TLR4 plays a key role in mediating pro-inflammatory responses, such as pro-inflammatory cytokine production in the CNS, when bound to various ligands including LPS.

Cholesterol is stored as cholesteryl esters. For cholesterol esterification, there are two Acyl-CoA:Cholesterol Acyltransferase (ACAT) genes, SOAT1 and SOAT2, encoding two homologous but distinct enzymes, ACAT1 and ACAT2. Both enzymes use long-chain fatty acyl-CoAs and sterols with 3-beta-OH, including cholesterol and various oxysterols as their substrates. ACAT1 is the major cholesterol storage enzyme in the brain. Both compound K-604 (Ki=0.5 μM) and compound F12511 (Ki=0.04 μM; US 2006/0135785) are high-affinity, ACAT1-specific, small-molecule inhibitors. Both inhibitors had passed phase I clinical safety tests for treating cardiovascular disease. K-604 is rather hydrophilic, while F12511 is extremely hydrophobic and both inhibitors tightly bind to ACAT1. F12511 preferentially inhibits ACAT1 but it also inhibits ACAT2 (Ki=0.11 μM).

Accordingly, needed in the art are new inhibitors that are selective, potent inhibitors of ACAT1.

### SUMMARY OF THE INVENTION

This invention provides a compound, designated herein as F24, having the structure:

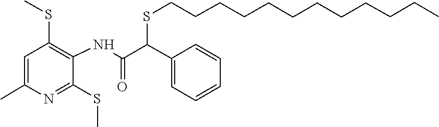 [SMILES: CCCCCCCCCCCCSC(C(=O)Nc1c(SC)cc(C)nc1SC)c1ccccc1]

or a derivative, pharmaceutically acceptable salt, stereoisomer, isotopic variant, N-oxide, or solvate thereof. The compound may be formulated in a pharmaceutical composition, which includes the compound in admixture with a pharmaceutically acceptable excipient or encapsulated in nanoparticles with an outer lipid envelope composed of distearoyl-sn-glycero-3-phosphoethanolamine-poly(ethylene glycol) and phosphatidylcholine.

This invention also provides a method for treating a subject suffering from, or diagnosed with a disease, disorder, or condition mediated by ACAT1 by administering to the subject an effective amount of F24 to treat the subjects disease, disorder, or condition. In some aspects, the disease, disorder, or condition comprises hypercholesterolemia, atherosclerosis, diet-induced obesity, glioma, a neurodegenerative disease, a neuroinflammatory disorder, or traumatic brain injury. In other aspects, the compound is administered intravenously or intraperitoneally. In further aspects, the method further includes administering an additional active ingredient.

### BRIEF DESCRIPTION OF THE DRAWINGS

FIG. 1 shows ACAT inhibition in forebrain, cerebellum, liver, adrenals gradually after a single IV of nanoparticle F12511 or nanoparticle F24. Shown is the efficacy of F12511 vs. F24 encapsulated in nanoparticle and IV delivered to mice. Four hours after IV, various tissues were isolated to monitor ACAT inhibition measurement. Mice were perfused before brains were isolated. N=2-3 WT mice/group. Statistical analysis, 2-way ANOVA. **P<0.01; ***P<0.001.

### DETAILED DESCRIPTION OF THE INVENTION

A high-affinity Acyl-CoA:Cholesterol Acyltransferase 1 (ACAT1) inhibitor is provided herein. The ACAT1 inhibitor of this invention, designated herein as “F24,” has the following structure:

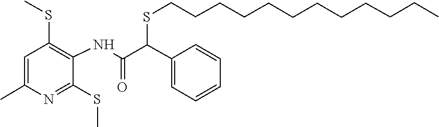 [SMILES: CCCCCCCCCCCCSC(C(=O)Nc1c(SC)cc(C)nc1SC)c1ccccc1]

Advantageously, F24 has an EC₅₀ of 6.0 nM in human cells and is brain-permeable when delivered via a nanoparticle. Accordingly, this invention provides the F24 compound, and derivatives thereof, as well as pharmaceutical compositions and nanoparticles including the F24 compound and methods of using the same in the treatment of a subject suffering from, or diagnosed with a disease, disorder, or medical condition mediated by ACAT1.

One aspect of this invention provides the F24 compound or a derivative thereof, and pharmaceutically acceptable salts, stereoisomers, isotopic variants, N-oxides, or solvates thereof. The term “pharmaceutically acceptable” means approved or approvable by a regulatory agency of Federal or a state government or the corresponding agency in countries other than the United States, or that is listed in the U.S. Pharmcopoeia or other generally recognized pharmacopoeia for use in animals, and more particularly, in humans.

A “pharmaceutically acceptable salt” is intended to mean a salt of a free acid or base of F24 or a derivative thereof that is non-toxic, biologically tolerable, or otherwise biologically suitable for administration to a subject. It should possess the desired pharmacological activity of the parent compound, i.e., ACAT1 inhibition. See, generally, Paulekuhn, et al. (2007) J. Med. Chem. 50:6665-72, Berge et al. (1977) J. Pharm. Sci. 66:1-19, and Handbook of Pharmaceutical Salts, Properties, Selection, and Use (2002) Stahl & Wermuth, Eds., Wiley-VCH and VHCA, Zurich. Examples of pharmaceutically acceptable salts are those that are pharmacologically effective and suitable for contact with the tissues of subjects without undue toxicity, irritation, or allergic response. F24 or derivatives thereof may possess a sufficiently acidic group, a sufficiently basic group, or both types of functional groups, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

For compounds containing a basic nitrogen, the desired pharmaceutically acceptable salt may be prepared by any suitable method available in the art. For example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

For compounds containing a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-D-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

“Stereoisomer” refers to both “diastereomers,” i.e., compounds that are not mirror images of one another, and “enantiomers,” compounds that are non-superimposable mirror images of each. When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” are also within the scope of this invention and refer to compounds that are interchangeable forms of a particular compound structure, and that vary in the displacement of hydrogen atoms and electrons. Thus, two structures may be in equilibrium through the movement of r electrons and an atom (usually H). For example, enols and ketones are tautomers because they are rapidly interconverted by treatment with either acid or base.

In addition, F24 or a derivative thereof may be in the form of a hydrate, solvate, or polymorph, and mixtures thereof. Solvates include those formed from the interaction or complexation of a compound with one or more solvents, either in solution or as a solid or crystalline form. In some aspects, the solvent is water and the solvates are hydrates. In addition, F24 or a derivative thereof, or a pharmaceutically acceptable salt thereof may be obtained as co-crystals.

The invention also provides pharmaceutically acceptable prodrugs of F24 or a derivative thereof, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to F24 or a derivative thereof). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs” (1985) Ed. H. Bundgaard, Elsevier.

Exemplary prodrugs include compounds having an amino acid residue, or a polypeptide chain of two or more (e.g., two, three or four) amino acid residues, covalently joined through an amide or ester bond to a free amino, hydroxyl, or carboxylic acid group. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups as amides or alkyl esters. Examples of amides include those derived from ammonia, primary C1-6 alkyl amines and secondary di(C1-6 alkyl) amines. Secondary amines include 5- or 6-membered heterocycloalkyl or heteroaryl ring moieties. Examples of amides include those that are derived from ammonia, C1-3 alkyl primary amines, and di(C1-2 alkyl)amines. Examples of esters of the invention include C1-7 alkyl, C5-7 cycloalkyl, phenyl, and phenyl(C1-6 alkyl) esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Fleisher et al. (1996) Adv. Drug Delivery Rev. 19:115-130. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in Robinson et al. (1996) J. Med. Chem. 39(1):10-18. Free amines can also be derivatized as amides, sulfonamides or phosphonamides. All of these prodrug moieties may incorporate groups including ether, amine, and carboxylic acid functionalities.

As demonstrated herein, F24 potently inhibits ACAT1 activity in vivo. Accordingly, this invention further provides a method for treating a subject suffering from, or diagnosed with a disease, disorder, or medical condition mediated by ACAT1. In the treatment method according to the invention, an effective amount of F24 and/or a derivative thereof is administered to a subject suffering from or diagnosed as having such a disease, disorder, or condition. An “effective amount” means an amount or dose sufficient to reduce the activity of ACAT1 by at least 60%, 70%, 80%, 90%, 95%, 99% or 100% and/or generally bring about the desired therapeutic or prophylactic benefit in subjects in need of such treatment for the designated disease, disorder, or condition. Effective amounts or doses of F24 and/or a derivative thereof may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the subject's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Subjects may, however, require intermittent treatment on a long-term basis upon any recurrence of symptoms. In the context of this invention, a subject can be any mammal including human, companion animals (e.g., dogs or cats), livestock (e.g., cows, sheep, pigs, or horses), or zoological animals (e.g., monkeys). In particular aspects, the subject is a human.

Examples of diseases, disorders, or conditions that can be treated with F24 and/or a derivative thereof include, but are not limited to, hypercholesterolemia, atherosclerosis, diet-induced obesity, glioma, a neurodegenerative disease, a neuroinflammatory disorder (e.g., acute disseminated encephalomyelitis, acute optic neuritis, transverse myelitis or neuromyelitis optica), or traumatic brain injury. Examples of diseases that include forms of neurodegeneration include, but are not limited to, acute neurodegeneration, such as intracranial brain injuries, such as stroke, diffuse and local brain injuries, epidural, subdural and subarachnoid haemorrhage, and chronic neurodegeneration, such as Alzheimer's disease, Huntington's disease, vascular dementia, frontotemporal dementia Parkinson's disease, tauopathy, multiple sclerosis and Amyotrophic lateral sclerosis (ALS).

In addition to F24 and/or a derivative thereof, the subject may be treated with additional active ingredients in the treatment of the above conditions. The additional active ingredients may be co-administered separately with F24 or a derivative thereof or included with F24 or a derivative thereof in a pharmaceutical composition according to the invention. Additional active ingredients are those that are known or discovered to be effective in the treatment of conditions, disorders, or diseases mediated by ACAT1 activity, such as another ACAT1 modulator or a compound active against another target associated with the particular condition, disorder, or disease. The combination may serve to increase efficacy (e.g., by including in the combination a compound potentiating the potency or effectiveness of F24 or a derivative thereof), decrease one or more side effects, or decrease the required dose of F24 or a derivative thereof. By way of illustration, when F24 or a derivative thereof is used in the treatment of a neurodegenerative disease or condition, F24 or a derivative thereof may be administered with an additional active ingredient that targets misfolded/aggregated protein/peptide such as amyloid β in AD, tau in AD, α-synuclein in PD, and frontotemporal dementia, and huntington in HD. Examples of such additional active ingredient of use in combination with F24 or a derivative thereof include, but are not limited to, β-secretase inhibitors/modulators (e.g., AZD3293, CTS-21166, E2609, HPP854, LY2886721, MK-8931, PF-05297909, RG7129 or TK-070), γ-secretase modulators (e.g., LY-411575, LY-450139, begacestat, ELN-475516, BMS-708163, MRK-003, CHF5074 or R04929097), proteasome inhibitors (e.g., Bortezomib), small molecule activators of the unfolded protein response (e.g., Celastrol), Hsp90 inhibitors (e.g., Geldanamycin), small molecule Hsc70 inhibitors (e.g., YM-01), deubiquitination enzyme inhibitors (e.g., siRNA targeting Usp14), epigallocatechin-3-gallate, variants of Hsp104 disaggregase, glutathione ethyl ester, and antibodies (e.g., anti-oligomeric amyloid 3 antibody, anti-tau antibody, anti-α-synuclein antibody or anti-huntington antibody).

F24 or a derivative thereof may be used alone, or in combination with one or more additional active ingredients, to formulate pharmaceutical compositions of the invention. A pharmaceutical composition of the invention includes an effective amount of F24 and/or a derivative thereof in admixture with pharmaceutically acceptable excipient. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of F24 and/or a derivative thereof may be prepared using suitable pharmaceutical excipients and compounding techniques known or that become available to those skilled in the art. The compositions may be administered in the inventive method by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, F24 or a derivative thereof can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include F24 or a derivative thereof mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

F24 or a derivative thereof may also be administered by non-oral routes. For example, the compositions may be formulated for rectal administration as a suppository. For parenteral use, including intravenous, intramuscular, intraperitoneal, or subcutaneous routes, F24 or a derivative thereof may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering F24 or a derivative thereof may utilize a patch formulation to affect transdermal delivery.

F24 or a derivative thereof may alternatively be administered in the method of this invention by inhalation, via the nasal or oral routes, e.g., in a spray formulation also containing a suitable carrier.

In some aspects, F24 or a derivative thereof is selectively delivered to the brain. For the purposes of the present invention, “selective delivery to the brain” or “selectively delivered to the brain” is intended to mean that the compound is administered directly to the brain of the subject (e.g., by a shunt or catheter; see, e.g., US 2008/0051691), to the perispinal space of the subject without direct intrathecal injection (see, e.g., U.S. Pat. No. 7,214,658), or in a form which facilitates delivery across the blood brain barrier thereby reducing potential side effects associated with ACAT1 inhibition in other organs or tissues. In this regard, formulation of the compound into a nanoparticle in the presence of a stabilizer allows passage of the blood brain barrier without affecting other organs with the agent. See, e.g., U.S. Pat. No. 7,402,573, incorporated herein by reference in its entirety.

It has been shown that exosomes (i.e., natural transport nanovesicles in the range of 40-100 nm), which express Lamp2b fused to the neuron-specific rabies viral glycoprotein (RVG) peptide, can deliver siRNA specifically to neurons, microglia, and oligodendrocytes in the brain, thereby resulting in specific gene knockdown (Alvarez-Erviti, et al. (2011) Nature Biotechnol. 29:341-345). Accordingly, in an alternative aspect, F24 or a derivative thereof is delivered to the brain via an exosome, in particular an exosome modified with a moiety that targets cells of the brain. Exosomes of use in this invention can be prepared by methods such as those described by, e.g., Sun, et al. (2010) Mol. Ther. 18:1606-1614. Likewise, therapeutic agents can be encapsulated within exosomes by methods such as incubating the therapeutic agent with an exosome preparation in saline at room temperature for several minutes, and separating the exosomes from unencapsulated drug and debris, e.g., by sucrose gradient separation. As described in the art, moieties that target cells of the brain include peptides that target cells of the brain (e.g., neurons, microglia and/or oligodendrocytes) as well as other targeting agents such as lipopolysaccharide, which has a high affinity for surface markers on microglia (Chow, et al. (1999) J. Biol. Chem. 274:10689-10692). Targeting peptides include, e.g., the RVG peptide, which may be fused to membrane bound proteins, e.g., Lamp2b (Lysosome-associated membrane protein 2b) to facilitate integration into the exosome.

In another alternative aspect, F24 or a derivative thereof is delivered intranasally via an exosome. Curcumin or Stat3 inhibitor, JSI-124 (cucurbitacin I), delivered via exosomes to the brain via the nasal route has been shown to accumulate in microglia and inhibit lipopolysaccharide (LPS)-induced microglial cell activation, delay experimental autoimmune encephalomyelitis (EAE) disease, and inhibit tumor growth in vivo (Zhuang, et al. (2011) Mol. Ther. 19:1769-1779). It is posited that transport occurs along the olfactory pathway and likely involves extracellular bulk flow along perineuronal and/or perivascular channels, which allows for delivering drugs directly to the brain parenchyma. Delivery along the extraneuronal pathway is likely not receptor-mediated and requires only minutes for a drug to reach the brain; whereas, delivery via an intraneuronal pathway along the primary olfactory sensory neurons involves axonal transport and requires several days for the drug to reach different areas of the brain. Therefore, in certain aspects, F24 or a derivative thereof is delivered to the brain, in particular microglia, by being encapsulated within exosomes and subsequent intranasal administration.

In one aspect, F24 and/or a derivative thereof is encapsulated in a nanoparticle. A nanoparticle of this invention denotes a sphere with a mean diameter of less than 300 nm, which has a circular, unilamellar lipid wall or envelope and a central component or core that contains F24 and/or a derivative thereof, and optionally one or more additional active ingredients. In certain aspects, the nanoparticles of this invention have at least one dimension in the range of about 10 nm to about 300 nm, including any integer value between 1 nm and 300 nm (including about 20, 30, 40, 50, 60, 70, 80, 90, 100, 200, 250, and 300). In certain aspects, the nanoparticles have at least one dimension that is about 15 nm to 250 nm. Particle size can be determined using any method known in the art, including, but not limited to, sedimentation field flow fractionation, photon correlation spectroscopy, disk centrifugation, dynamic light scattering, and electron microscopy.

In another aspect, F24 and/or a derivative thereof are encapsulated in a stealth nanoparticle. The nanoparticles of this invention are “stealth” in that they have the ability to not be detected and then sequestered and/or degraded, or to be hardly detected and then sequestered and/or degraded, and/or to be detected and then sequestered and/or degraded late, by the immune system of the host to which they are administered. In this respect, the nanoparticles exhibit long circulating properties which provide them with a half-life time in the blood compartment of greater than 2, 4, 6, 8, 10, or 12 hours thereby making it possible to allow a significant portion of the nanoparticle (approximately 0.5% (Saucier-Sawyer et al. (2015) J. Drug Target. 23(7-8):736-49) to enter the brain. Further, the nanoparticles according to the present invention can carry a relatively high content of active ingredient through a subject thereby providing reduced toxicity compared with free drug in solution.

Stealth nanoparticles may be composed of a core which is liquid or semi-liquid at ambient temperature, and a lipid envelope composed of phosphatidylcholine (PC) and a biodegradable phospholipid, preferably a pegylated phospholipid, most preferably distearoyl-sn-glycero-3-phosphoethanolamine-poly(ethylene glycol)ₓ (DSPE-PEGx), in which x represents the size of the PEG molecule in g/mol. In some aspects, the molar mass of the PEG component is in the range of about 100 to about 20,000 g/mol, including about 500 g/mol, about 600 g/mol, about 700 g/mol, about 800 g/mol, about 900 g/mol, about 1000 g/mol, about 2000 g/mol, about 3000 g/mol, about 5000 g/mol, about 10,000 g/mol, about 15,000 g/mol, and about 20,000 g/mol. In certain aspects, the molar mass of the PEG component is greater than or equal to 1000 g/mol, preferably greater than or equal to 2000 g/mol. In some aspects, DSPE-PEG₁₀₀₀, DSPE-PEG₂₀₀₀, DSPE-PEG₃₀₀₀ or DSPE-PEG₅₀₀₀ is used. In particular aspects, DSPE-PEG₂₀₀₀ or DSPE-PEG₅₀₀₀ is used. Preferably, the nanoparticles include between 20 mM and 40 mM, preferably between 25 mM and 35 mM, most preferably 30 mM of the pegylated phospholipid (e.g., DSPE-PEG₂₀₀₀ or DSPE-PEG₅₀₀₀).

The phosphatidylcholine used in the preparation of the nanoparticles may be purified to at least 95%, 96%, 97%, 98%, 99% or 100% homogeneity. In this respect, the phosphatidylcholine has less than 5%, 4%, 3%, 2%, 1%, 0.5%, or 0.1% impurities such as other phosphatides (e.g., phosphatidylethanolamine, phosphatidylserine, phosphatidic acid, and phosphatidylinositol), triglycerides, fatty acids, or carbohydrates. In particular aspects, the nanoparticles include between 2 mM and 10 mM, preferably between 4 mM and 8 mM, most preferably 6 mM phosphatidylcholine.

The addition of between 3 mM and 6 mM phosphatidylcholine to DSPE-PEG₂₀₀₀ prior to nanoparticle formation yields the incorporation of about 0.9 mM F24. Accordingly, the nanoparticles may encapsulate between 0.1 mM and 5 mM, preferably between 0.5 mM and 3 mM F24. In addition, the ratio of F24 or a derivative thereof to phosphatidylcholine may be in the range of about 0.3:1 to 2:1, or more preferably 2:1. Further, the DSPE-PEG to PC ratio may be in the range of 30:3 to 30:12, or more preferably 30:6. Moreover, in certain aspects, the concentration of PC is greater than or equal to 6 mM. Advantageously, nanoparticle composed of 30 mM DSPE-PEG and 6 mM PC can provide 3 mM DSPE-PEG and 0.6 mM PC in the blood of a subject immediately upon delivery of the nanoparticle.

While the nanoparticle can be composed solely of a pegylated phospholipid, phosphatidylcholine and F24 and/or a derivative thereof, the nanoparticle may further be modified to include a targeting ligand. By “targeting ligand” is meant a molecule that targets a physically associated molecule or complex to a targeted cell or tissue. As used herein, the term “physically associated” refers to either a covalent or non-covalent interaction between two molecules. A “conjugate” refers to the complex of molecules that are covalently bound to one another. For example, the complex of a lipid covalently bound to a targeting ligand can be referred to as a lipid-targeting ligand conjugate.

Alternatively, the targeting ligand can be non-covalently bound to a lipid. “Non-covalent bonds” or “non-covalent interactions” do not involve the sharing of pairs of electrons, but rather involve more dispersed variations of electromagnetic interactions, and can include hydrogen bonding, ionic interactions, Van der Waals interactions, and hydrophobic bonds.

Targeting ligands can include, but are not limited to, small molecules, peptides, lipids, sugars, oligonucleotides, hormones, vitamins, antigens, antibodies or fragments thereof, specific membrane-receptor ligands, ligands capable of reacting with an anti-ligand, fusogenic peptides, nuclear localization peptides, or a combination of such compounds. Non-limiting examples of targeting ligands include transferrin, OX26, melanotransferrin, insulin, ApoE, leptin, thiamine or vitamin B12, rabies virus glycoprotein (RVG), cell penetrating peptides such as TAT peptide, and monoclonal and polyclonal antibodies directed against cell surface molecules. The targeting ligand can be covalently bound to the lipids of the nanoparticle using techniques known in the art (Ishida et al. (1999) FEBS Lett. 460:129-133; Perouzel et al. (2003) Bioconjug. Chem. 14:884-898).

In a further alternative aspect, F24 and/or a derivative thereof is encapsulated in human serum albumin (HSA) nanoparticles to enhance solubility and bioavailability of the inhibitor. Such nanoparticles can be fabricated via unfolding of HSA in appropriate solution to expose more hydrophobic domains and consequent self-assembling into nanoparticles with the ACAT inhibitor. See Zhou, et al. (2016) Anticancer Res. 36(4):1649-56; Ding, et al. (2014) AAPS PharmSciTech. 15(1):213-22.

While certain aspects of this invention embrace in vivo applications, in vitro use of F24 or a derivative thereof is also contemplated for examining the effects of ACAT1 inhibition on cell expression of Acat1 or activity of ACAT1. In addition to treatment, F24 or a derivative thereof also finds application in monitoring the phenotypic consequences (e.g., expression or activity of cytokines and chemokines, etc.) of neuroinflammation.

The invention is described in greater detail by the following non-limiting examples.

### Example 1: F24 Potently Inhibits ACAT1 In Vitro

F24 was custom synthesized by WUXI Chemistry Service Unit in China. Based on HPLC/MS and NMR profiles, purity of F24 was 98% and in stereospecificity. Organic solvents were from Fisher Scientific.

Chinese Hamster Ovary (CHO) cells stably expressing human ACAT1 or human ACAT2 were used as described in the literature (Chang et al. (2000) J. Biol. Chem. 275(36):28083-92). Cells were placed in 10% serum for two days at 37° C. and further incubated with various concentrations of F24 as a stock solution in DMSO for 2 hours. Subsequently, a complex of [3H]-oleic acid and BSA (serving as precursor of oleyl CoA as substrate of ACAT) was fed to the cells for 2 hours. Cells were extensively washed and cellular lipids were extracted with organic solvents. Radioactivity of cholesteryl [3H]-oleate was separated by thin-layer chromatography and determined by scintillation counting. The EC₅₀ value of F24 was calculated using the Prism 9 program. This analysis indicated that F24 exhibited was nearly seven-fold more potent than F12511 in inhibiting ACAT1 (6 nM vs. 39 nM) and five-fold more potent than F12511 in inhibiting human ACAT2 (11 nM vs. 55 nM).

### Example 2: F24 Potently Inhibits ACAT1 In Vivo

Nanoparticle Formation. Nanoparticles were prepared according to a general method (Gülçür, et al. (2013) Drug Deliv. Transl. Res. 3(6):562-574; Jhaveri & Torchilin (2014) Front. Pharmacol. 5:77) with modifications. Using a clean glass tube (9 mL capacity), 60 mg of DSPE-PEG₂₀₀₀ (Laysan Bio, Inc (mPEG-DSPE, MW 2,000)) was dissolved in 500 μL of ethanol (EtOH) for a working concentration of 60 mM. L-α-Phosphatidylcholine (PC, Sigma-Aldrich) (dissolved in chloroform) was added at working concentrations ranging from 0 to 12 mM to the DSPE-PEG₂₀₀₀ solution while vortexing. F12511 and F24 were dissolved in 500 μL of ethanol then added to the DSPE-PEG₂₀₀₀/PC mixture while vortexing. The final solution contained concentrations of 30 mM DSPE-PEG₂₀₀₀, 0-6 mM PC, and 9 mM F12511 or 0.9 mM F24. The final solution was then lyophilized under refrigeration overnight to remove organic solvent. The sample was re-suspended in 1 mL of phosphate-buffered saline (1×) (PBS) and vortexed until the sample was in suspension. This step took approximately 1 hour as the sample needed to rest between repeated vortexing to prevent excessive foaming. Once in fine suspension, the sample was purged with nitrogen, capped, wrapped with parafilm, and bath sonicated (Branson 2510 model) at 4° C. for 2-4 rounds at 20 minutes per round. After sonication, nanoparticles were transferred to sterile Eppendorf tubes and centrifuged at 12,000 rpm for 5 minutes to remove unincorporated materials. The supernatant as well as the pellet were collected for chemical analysis. In some experiments, the nanoparticles were loaded onto a 5 mL SEPHAROSE® (crosslinked agarose) CL-4B column. SEPHAROSE® CL-4B contains beads with particle size at 45-165 μm. The column (approximately 28 mm circumference) was first equilibrated with PBS at room temperature, then loaded with the 1 mL of sample, eluted with PBS and fractions of 500 μL to 1 mL were collected. This method was used to assure that compounds encapsulated in nanoparticles did not appear in the exclusion volume of the SEPHAROSE® CL-4B column.

Nanoparticle Analysis. To confirm the concentration of compound and DSPE-PEG₂₀₀₀/PC in the nanoparticles, 10 μL of each sample component were loaded onto a thin layer chromatography (TLC) plate (Analtech Silica gel HL). The content of compound was determined by extrapolation from a standard curve of a concentration gradient of the respective compound produced in the same TLC plate. The plate was stained with iodine and quantified using ImageJ. The TLC/iodine stain method detected compound, and DSPE-PEG₂₀₀₀/PC separately, thus allowing for encapsulation efficiency determination.

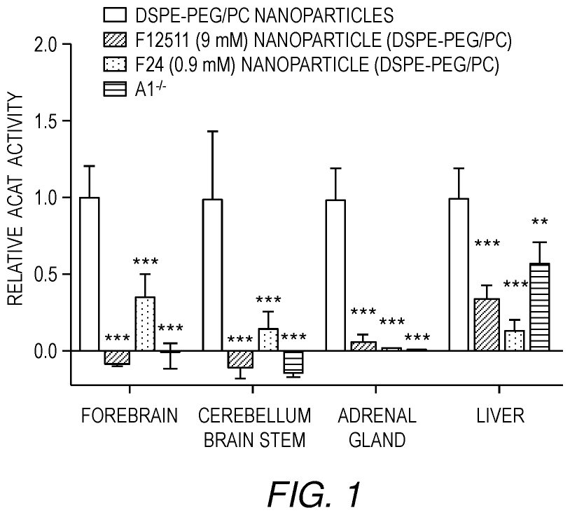

Delivery of F24 in Mice via Nanoparticles. To determine in vivo activity, F12511 and F24 were delivered IV to mice via stealth nanoparticles (nanoparticle F12511 and nanoparticle F24). Adult C57BL/6 mice were given single IV of nanoparticle F12511 (at high dose, 9 mM) or nanoparticle F24 (at low dose, 0.9 mM). Four hours after IV delivery, different tissues were isolated and the percent of ACAT inhibition was assessed. The results (FIG. 1) showed that a low dose of F24 provided 70% ACAT inhibition, indicating that F24 is brain-permeable and very potent at inhibiting ACAT activity in the brain.

What is claimed is:

1. A compound having the structure: 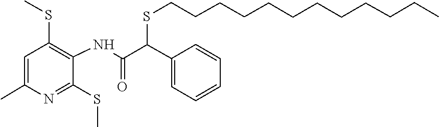 [SMILES: CCCCCCCCCCCCSC(C(=O)Nc1c(SC)cc(C)nc1SC)c1ccccc1]
or a derivative, pharmaceutically acceptable salt, stereoisomer, isotopic variant, N-oxide, or solvate thereof.

2. A pharmaceutical composition comprising the compound of claim 1 in admixture with a pharmaceutically acceptable excipient.

3. A nanoparticle composition comprising nanoparticles having an outer lipid envelope and a core, the outer lipid envelope comprising distearoyl-sn-glycero-3-phosphoethanolamine-poly(ethylene glycol) and phosphatidylcholine, the core comprising the compound of claim 1.

4. A method for treating a subject suffering from, or diagnosed with a disease, disorder, or condition mediated by Acyl-CoA:Cholesterol Acyltransferase 1 (ACAT1) comprising administering to the subject an effective amount of the compound of claim 1 to treat the subjects disease, disorder, or condition.

5. The method of claim 4, wherein the disease, disorder, or condition comprises hypercholesterolemia, atherosclerosis, diet-induced obesity, glioma, a neurodegenerative disease, a neuroinflammatory disorder, or traumatic brain injury.

6. The method of claim 4, wherein the compound is administered intravenously or intraperitoneally.

7. The method of claim 4, further comprising administering an additional active ingredient.
